(19) United States
(12) Patent Application Publication
(10) Pub. No.: US 2014/0238112 A1
(43) Pub. Date: Aug. 28, 2014

(54) SYSTEM FOR MONITORING FLUID IN A CONTAINER

(71) Applicants: Alexander Himanshu Amin, Solon, OH (US); Ilan Eric Ponsky, Moreland Hills, OH (US); Himanshu Subhash Amin, Solon, OH (US)

(72) Inventors: Alexander Himanshu Amin, Solon, OH (US); Ilan Eric Ponsky, Moreland Hills, OH (US); Himanshu Subhash Amin, Solon, OH (US)

(21) Appl. No.: 13/772,300

(22) Filed: Feb. 20, 2013

Related U.S. Application Data
(60) Provisional application No. 61/600,863, filed on Feb. 20, 2012.

(51) Int. Cl. G01N 33/00; G01F 23/00

(52) U.S. Cl. CPC G01N 33/00; G01F 23/00
USPC 73/61.41; 73/53.01

(57) ABSTRACT

This disclosure relates to systems and methods for monitoring attributes of a fluid within a container, and wirelessly transmitting fluid information to one or more remote deices.

### TECHNICAL FIELD

This disclosure generally relates to systems and methods that facilitate monitoring fluid in a container.

### BACKGROUND

Users purchase and store numerous products that contain fluids (e.g., milk, juice, water, detergent, soap, oils, beverages, alcohol, teas, coffee, etc.), and these fluids are held in containers. Oftentimes, the container is solid and non-transparent as well as stored in a location that is not readily viewable (e.g., closet, refrigerator, . . . ). Consequently, especially given the large number of containers in a household, office, store, etc., it becomes difficult to assess quantity of fluid in respective containers as well as quality of the fluids.

### SUMMARY

The following presents a simplified summary of the disclosure in order to provide a basic understanding of some aspects of the disclosure. This summary is not an extensive overview of the disclosure. It is intended to neither identify key or critical elements of the disclosure nor delineate any scope particular embodiments of the disclosure, or any scope of the claims. Its sole purpose is to present some concepts of the disclosure in a simplified form as a prelude to the more detailed description that is presented later.

In accordance with one or more embodiments and corresponding disclosure, various non-limiting aspects are described in connection with monitoring fluid level, quality, temperature, age, and other attributes.

The following description and the annexed drawings set forth certain illustrative aspects of the disclosure. These aspects are indicative, however, of but a few of the various ways in which the principles of the disclosure may be employed. Other advantages and novel features of the disclosure will become apparent from the following detailed description of the disclosure when considered in conjunction with the drawings.

### BRIEF DESCRIPTION OF THE DRAWINGS

FIGS. 1-3 illustrate example non-limiting system(s) for monitoring a fluid in a container in accordance with one or more implementations described herein.

FIG. 4 illustrates an example non-limiting interface for a monitoring system and sensor for monitoring a fluid in a container in accordance with one or more implementations described herein.

FIG. 5 illustrates an example non-limiting schematic representation of a container sidewall, and example system for monitoring a fluid in a container in accordance with one or more implementations described herein.

FIGS. 6-7 illustrate example methodologies for monitoring fluid in a container in accordance with one or more implementations described herein.

FIG. 8 is a block diagram representing an exemplary non-limiting computing system or operating environment in which the various embodiments may be implemented.

FIG. 9 is a block diagram representing an exemplary non-limiting networked environment in which the various embodiments can be implemented.

### DETAILED DESCRIPTION OVERVIEW

The innovation is now described with reference to the drawings, wherein like reference numerals are used to refer to like elements throughout. In the following description, for purposes of explanation, numerous specific details are set forth in order to provide a thorough understanding of this innovation. It may be evident, however, that the innovation can be practiced without these specific details. In other instances, well-known structures and devices are shown in block diagram form in order to facilitate describing the innovation.

By way of introduction, the subject matter disclosed in this disclosure relates to monitoring fluid in a container, and transmitting fluid information to remote devices. For example, fluid level, fluid quality, fluid age, fluid temperature, and other attributes are monitored and/or determined, and the associated fluid information is transmitted to one or more devices to apprise one or more users of status of the fluid and its container.

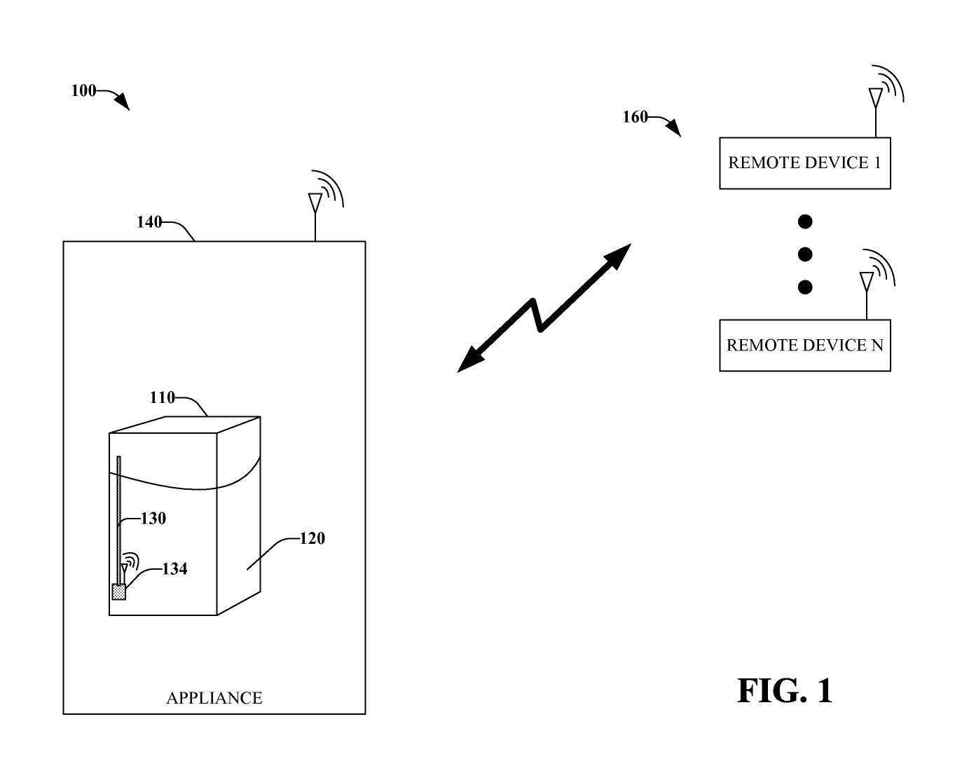

Referring now to the drawings, with reference initially to FIG. 1, a monitoring system 100 is shown that facilitates monitoring fluid level in a container 110 that holds a fluid 120 (e.g., milk, juice, water, detergent, soap, oils, beverages, alcohol, teas, coffee, etc.). The container 110 includes a sensor 130 that is operatively coupled to a sensing system 134 that wirelessly transits gather fluid information to an appliance 140 (e.g., refrigerator) and/or a set of remote devices 160 (e.g., cell phone, personal data assistant (PDA), tablet PC, personal computer (PC), desktop computer, automobile, television, e-book, gaming system . . . ). Aspects of the systems, apparatuses or processes explained in this disclosure can constitute machine-executable components embodied within machine(s), e.g., embodied in one or more computer readable mediums (or media) associated with one or more machines. Such components, when executed by the one or more machines, e.g., computer(s), computing device(s), virtual machine(s), etc. can cause the machine(s) to perform the operations described.

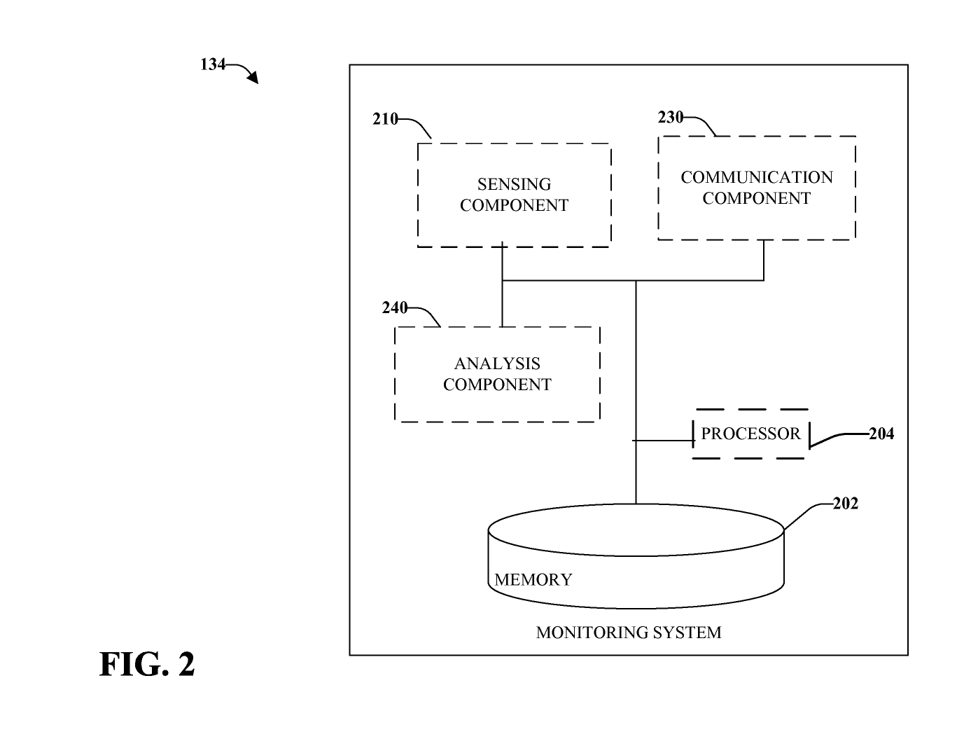

The sensor 130 in conjunction with the sensing system 134 can determine the various attributes of the fluid. For example, the sensor 134 can include multiple components that can measure fluid level, fluid temperature, fluid content, pH levels, viscosity, rancidity, density, particle content, etc. Any suitable type of sensor that can be employed in connection with facilitating detection, monitoring or measuring one or more of the foregoing attributes is contemplated. The sensing system 134 is described in greater detail in connection with FIGS. 2-4, and it analyzes data gathered from the sensor 130 as well as extrinsic information (e.g., ambient temperature, age of container or fluid, where purchased, when purchased, movement of container, location of container, etc.) in connection with generating detailed information regarding the fluid 120 and the container 110. The system 134 can transmit the detailed fluid information for example to the appliance 140 or the remote devices 160. It is to be appreciated that the appliance 140 can likewise forward transmit the fluid information to the remote devices 160.

In an embodiment, the remote devices 160 and/or the appliance 140 can interrogate the system 134 for real-time fluid information in addition to receiving broadcast information from the system 134.

FIG. 2 illustrates an example schematic representation of sensing system 134. System 134 can include memory 202 for storing computer executable components and instructions. A processor 204 can facilitate operation of the computer executable components and instructions by the system 134. A sensing component 210 can gather information regarding attributes of the fluid as noted above. The sensing component 210 can include anyone or more of a variety of fluid sensors (e.g., pH sensor, temperature sensor, viscosity sensor, particle sensor, rancidity sensor, contaminant sensor, quality sensor, bacteria sensor, etc.). Any suitable type(s) of sensor(s) (e.g., MEMS, semiconductor, biochip, electro-optical, inductive, chemical, photo-chemical, LED, potentio-metric, colorimeter, photo-detector, photodiode, strip, conductivity, photo-resistor, etc.) can be employed. An analysis component 240 analyzes the fluid data gathered by the sensing component 210, and makes a determination or inference regarding state or attributes of the fluid. The analysis component 240 can employ lookup tables, digital signatures, artificial intelligence, and the like to make the respective determinations or inferences. The analysis component 240 can generate fluid condition information that is transmitted to other devices by the communication component 230.

In one non-limiting exemplary implementation, analysis component 240 can automatically and dynamically analyze the fluid with use of a dynamic Bayesian network such as a Hidden Markov Model (HMM) using a Viterbi algorithm.

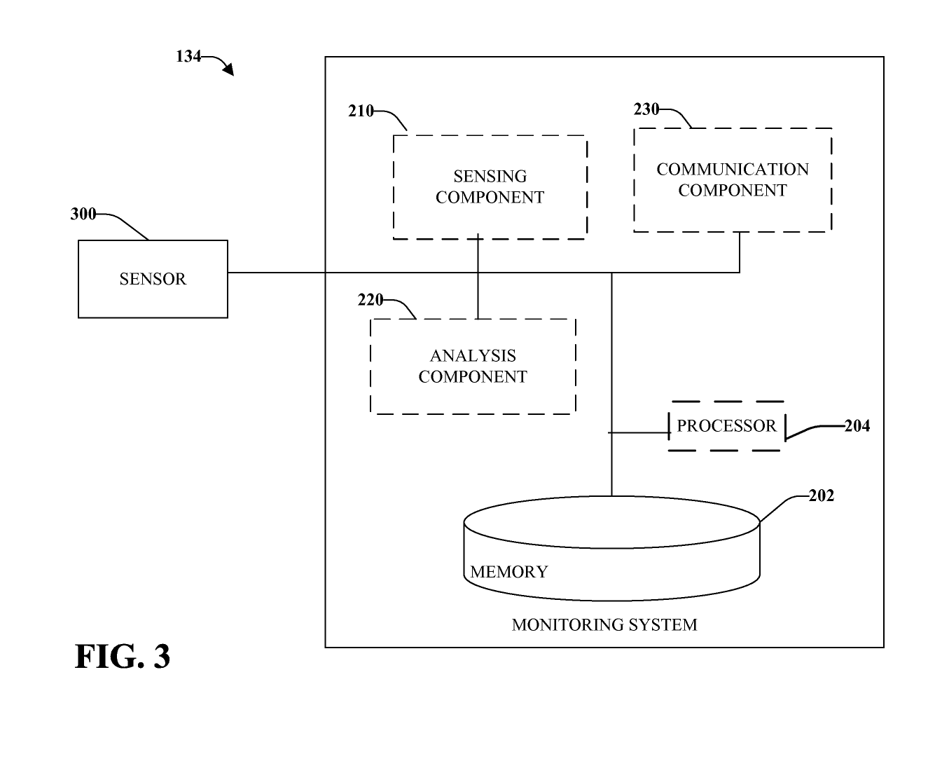

FIG. 3 illustrates an embodiment where the system 134 receives and analyzes fluid data gathered by an external fluid sensor.

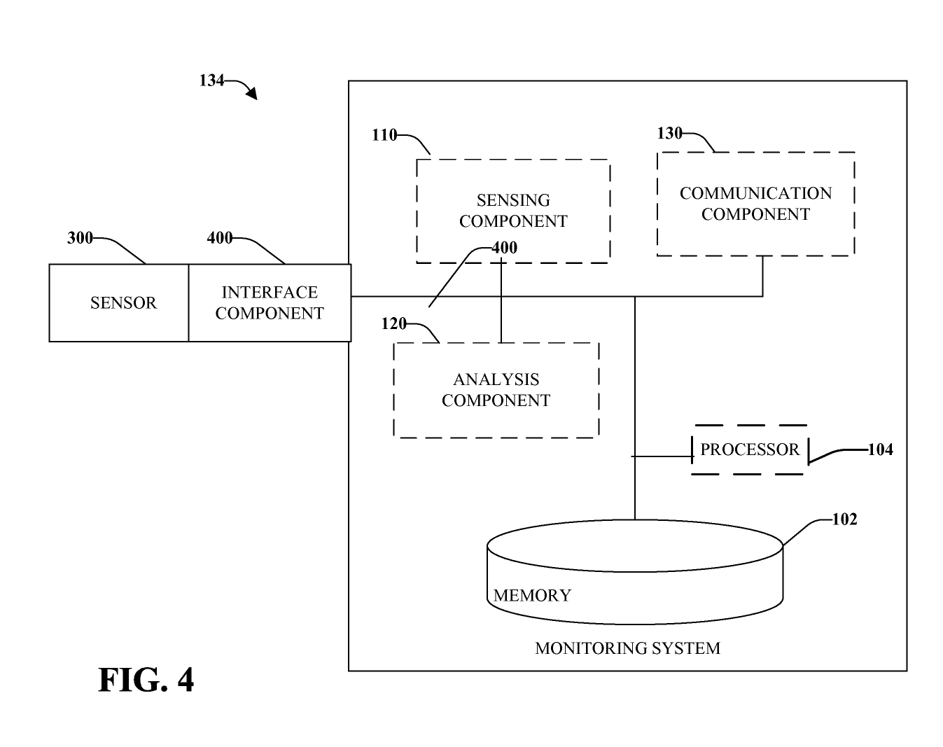

FIG. 4 illustrates am embodiment where the sensor 300 interfaces with the system 134 via an interface component 400. The interface component 400 can be an electro-mechanical interface that provides for the sensing system 134 to interface with the sensor 300. For example, the sensor 300 can be integrally formed within the housing of the container, and the interface 400 provides or operatively coupling an attachable/detachable embodiment of sensing system 134 to the sensor 300.

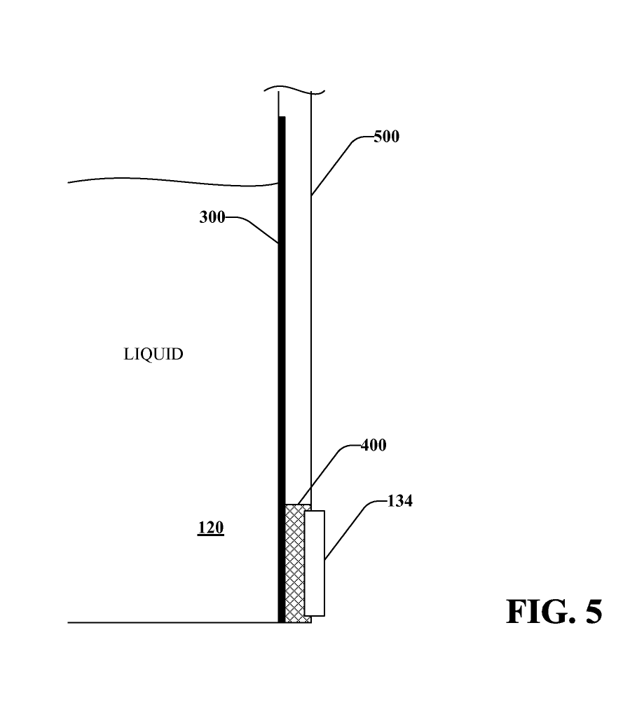

FIG. 5 schematically illustrates a sidewall 500 of the container 140. The sensor 300 is disposed along an interior wall of the container 140 and in direct contact with the fluid 120 as well as open space within the container 140. Interface 400 provides for coupling the sensor 300 to the externally disposed sensing system 134. It is to be appreciated that the system 134 can be detachable and re-useable in certain embodiments, or be permanently affixed to the container in other embodiments. In the re-useable embodiment, the system 134 can be stored and charged until ready for use by attaching to the interface 400 of a container. For example, the system 134 can be charged via wireless induction, standard induction, solar, battery, MEMS, RF, or any suitable means. Thus, once notified that a container is empty or near empty, the sensing system 134 can be removed from the interface for later use, and this stop collecting and transmitting information regarding fluid in the discarded container.

In an embodiment where the sensing system 134 is permanently attached, the system can be configured to stop collecting information/transmitting after one or more transmissions regarding the fluid falling below a pre-determined level (e.g., measurement or quality, etc.). In another implementation, the sensing system 134 can be deactivated manually, chemically, or electronically.

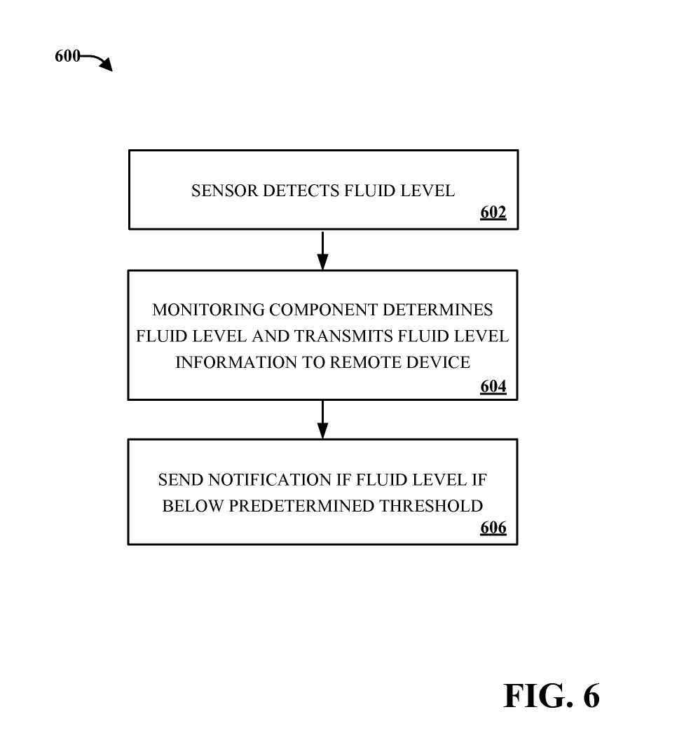

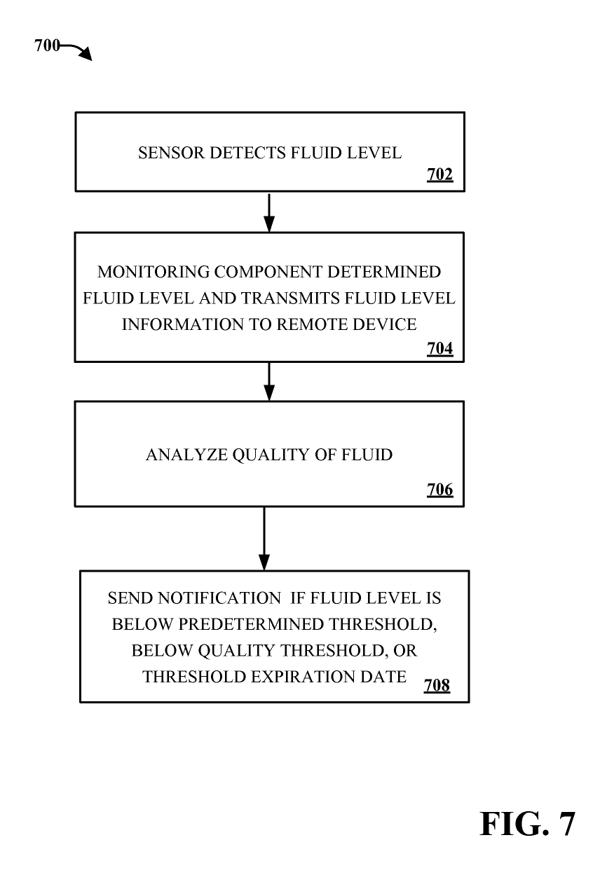

FIGS. 6 and 7 illustrate example methodologies or flow diagrams in accordance with certain aspects of this disclosure. While, for purposes of simplicity of explanation, the methodologies are shown and described as a series of acts, the disclosed subject matter is not limited by the order of acts, as some acts may occur in different orders and/or concurrently with other acts from that shown and described herein. For example, those skilled in the art will understand and appreciate that a methodology can alternatively be represented as a series of interrelated states or events, such as in a state diagram. Moreover, not all illustrated acts may be required to implement a methodology in accordance with the disclosed subject matter. Additionally, it is to be appreciated that the methodologies disclosed in this disclosure are capable of being stored on an article of manufacture to facilitate transporting and transferring such methodologies to computers or other computing devices.

Referring now to FIG. 6, presented is a flow diagram of an example fluid monitoring process. At 602, a sensor detects level of a fluid in a container. At 604, a monitoring component determines fluid level, and transmits fluid level information to a remote device. At 606, a notification is transmitted to one or more remote devices if the fluid level has fallen below a pre-determined threshold.

Referring now to FIG. 7, presented is a flow diagram of an example fluid monitoring process. At 702, a sensor detects level of a fluid in a container. At 704, a monitoring component determines fluid level, and transmits fluid level information to a remote device. At 706, quality of the fluid is analyzed. At 708, a notification is transmitted to one or more remote devices if the fluid level, fluid quality, or threshold expiration has passed a pre-determined threshold.

In view of the exemplary systems described above, methodologies that may be implemented in accordance with the described subject matter will be better appreciated with reference to the flowcharts of the various figures. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described in this disclosure. Where non-sequential, or branched, flow is illustrated via flowchart, it can be appreciated that various other branches, flow paths, and orders of the blocks, may be implemented which achieve the same or a similar result. Moreover, not all illustrated blocks may be required to implement the methodologies described hereinafter.

In addition to the various embodiments described in this disclosure, it is to be understood that other similar embodiments can be used or modifications and additions can be made to the described embodiment(s) for performing the same or equivalent function of the corresponding embodiment(s) without deviating there from. Still further, multiple processing chips or multiple devices can share the performance of one or more functions described in this disclosure, and similarly, storage can be affected across a plurality of devices. Accordingly, the invention is not to be limited to any single embodiment, but rather can be construed in breadth, spirit and scope in accordance with the appended claims.

### Example Operating Environments

The systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which may be explicitly illustrated in this disclosure.

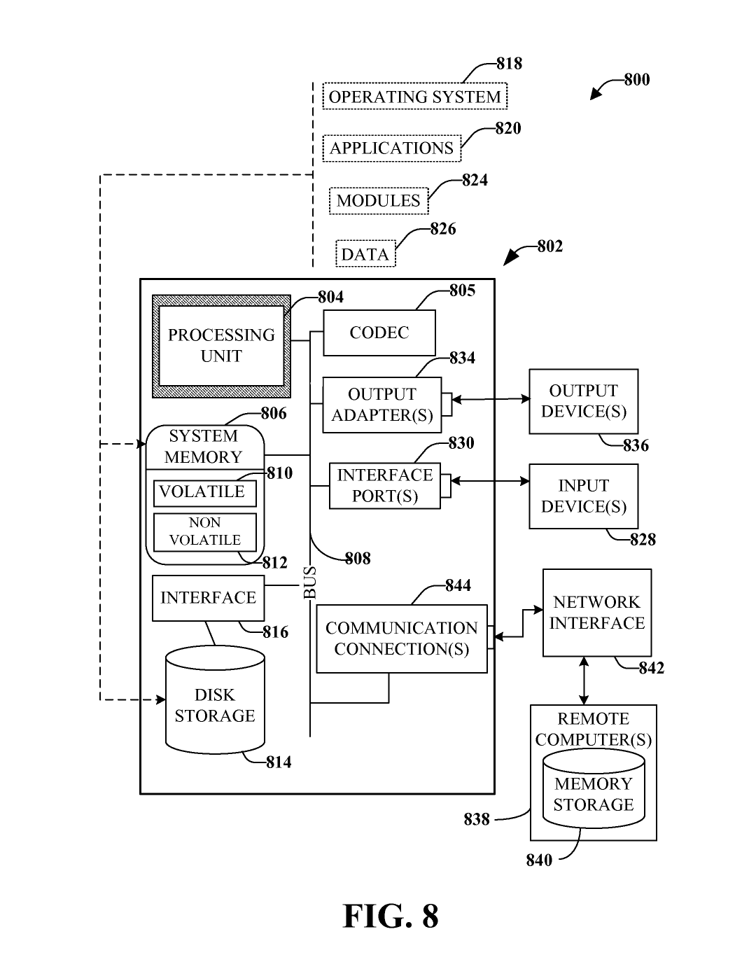

With reference to FIG. 8, a suitable environment 800 for implementing various aspects of the claimed subject matter includes a computer 802. The computer 802 includes a processing unit 804, a system memory 806, a codec 805, and a system bus 808. The system bus 808 couples system components including, but not limited to, the system memory 806 to the processing unit 804. The processing unit 804 can be any of various available processors. Dual microprocessors and other multiprocessor architectures also can be employed as the processing unit 804.

The system bus 808 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 806 includes volatile memory 810 and nonvolatile memory 812. The basic input/output system (BIOS), containing the basic routines to transfer information between elements within the computer 802, such as during start-up, is stored in non-volatile memory 812. In addition, according to present innovations, codec 805 may include at least one of an encoder or decoder, wherein the at least one of an encoder or decoder may consist of hardware, a combination of hardware and software, or software. Although, codec 805 is depicted as a separate component, codec 805 may be contained within non-volatile memory 812. By way of illustration, and not limitation, non-volatile memory 812 can include read only memory (ROM), programmable ROM (PROM), electrically programmable ROM (EPROM), electrically erasable programmable ROM (EEPROM), or flash memory. Volatile memory 810 includes random access memory (RAM), which acts as external cache memory. According to present aspects, the volatile memory may store the write operation retry logic (not shown in FIG. 8) and the like. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM.

Computer 802 may also include removable/non-removable, volatile/non-volatile computer storage medium. FIG. 8 illustrates, for example, disk storage 814. Disk storage 814 includes, but is not limited to, devices like a magnetic disk drive, solid state disk (SSD) floppy disk drive, tape drive, Jaz drive, Zip drive, LS-70 drive, flash memory card, or memory stick. In addition, disk storage 814 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 814 to the system bus 808, a removable or non-removable interface is typically used, such as interface 816.

It is to be appreciated that FIG. 8 describes software that acts as an intermediary between users and the basic computer resources described in the suitable operating environment 800. Such software includes an operating system 818. Operating system 818, which can be stored on disk storage 814, acts to control and allocate resources of the computer system 802. Applications 820 take advantage of the management of resources by the operating system through program modules 824, and program data 826, such as the boot/shutdown transaction table and the like, stored either in system memory 806 or on disk storage 814. It is to be appreciated that the claimed subject matter can be implemented with various operating systems or combinations of operating systems.

A user enters commands or information into the computer 802 through input device(s) 828. Input devices 828 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 804 through the system bus 808 via interface port(s) 830. Interface port(s) 830 include, for example, a serial port, a parallel port, a game port, and a universal serial bus (USB). Output device(s) 836 use some of the same type of ports as input device(s) 828. Thus, for example, a USB port may be used to provide input to computer 802, and to output information from computer 802 to an output device 836. Output adapter 834 is provided to illustrate that there are some output devices 836 like monitors, speakers, and printers, among other output devices 836, which require special adapters. The output adapters 834 include, by way of illustration and not limitation, video and sound cards that provide a means of connection between the output device 836 and the system bus 808. It should be noted that other devices and/or systems of devices provide both input and output capabilities such as remote computer(s) 838.

Computer 802 can operate in a networked environment using logical connections to one or more remote computers, such as remote computer(s) 838. The remote computer(s) 838 can be a personal computer, a server, a router, a network PC, a workstation, a microprocessor based appliance, a peer device, a smart phone, a tablet, or other network node, and typically includes many of the elements described relative to computer 802. For purposes of brevity, only a memory storage device 840 is illustrated with remote computer(s) 838. Remote computer(s) 838 is logically connected to computer 802 through a network interface 842 and then connected via communication connection(s) 844. Network interface 842 encompasses wire and/or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 844 refers to the hardware/software employed to connect the network interface 842 to the bus 808. While communication connection 844 is shown for illustrative clarity inside computer 802, it can also be external to computer 802. The hardware/software necessary for connection to the network interface 842 includes, for exemplary purposes only, internal and external technologies such as, modems including regular telephone grade modems, cable modems and DSL modems, ISDN adapters, and wired and wireless Ethernet cards, hubs, and routers.

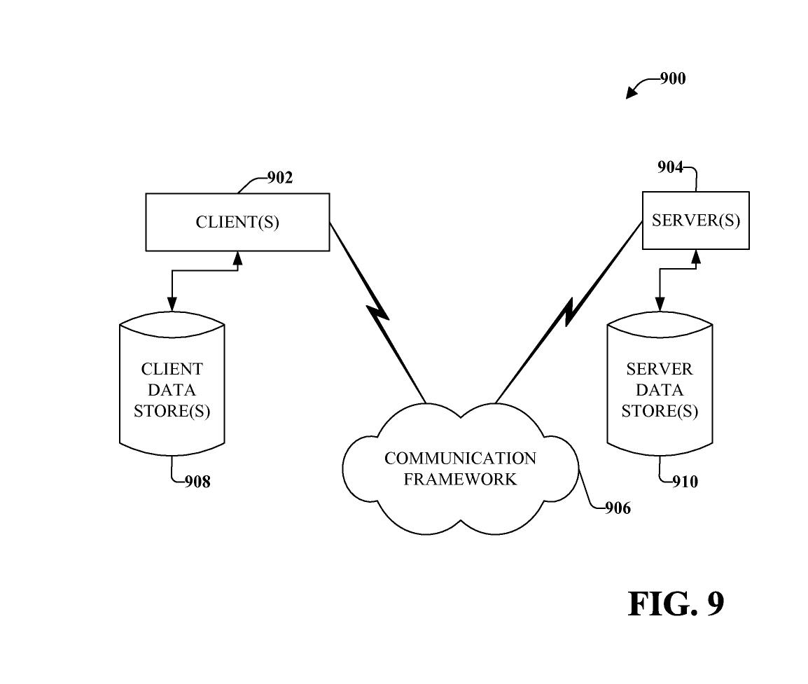

Referring now to FIG. 9, there is illustrated a schematic block diagram of a computing environment 900 in accordance with this disclosure. The system 900 includes one or more client(s) 902 (e.g., laptops, smart phones, PDAs, media players, computers, portable electronic devices, tablets, and the like). The client(s) 902 can be hardware and/or software (e.g., threads, processes, computing devices). The system 900 also includes one or more server(s) 904. The server(s) 904 can also be hardware or hardware in combination with software (e.g., threads, processes, computing devices). The servers 904 can house threads to perform transformations by employing aspects of this disclosure, for example. One possible communication between a client 902 and a server 904 can be in the form of a data packet transmitted between two or more computer processes wherein the data packet may include video data. The data packet can include a metadata, such as associated contextual information for example. The system 900 includes a communication framework 906 (e.g., a global communication network such as the Internet, or mobile network(s)) that can be employed to facilitate communications between the client(s) 902 and the server(s) 904.

Communications can be facilitated via a wired (including optical fiber) and/or wireless technology. The client(s) 902 include or are operatively connected to one or more client data store(s) 908 that can be employed to store information local to the client(s) 902 (e.g., associated contextual information). Similarly, the server(s) 904 are operatively include or are operatively connected to one or more server data store(s) 910 that can be employed to store information local to the servers 904.

In one embodiment, a client 902 can transfer an encoded file, in accordance with the disclosed subject matter, to server 904. Server 904 can store the file, decode the file, or transmit the file to another client 902. It is to be appreciated, that a client 902 can also transfer uncompressed file to a server 904 and server 904 can compress the file in accordance with the disclosed subject matter. Likewise, server 904 can encode video information and transmit the information via communication framework 906 to one or more clients 902.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described in this description can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described in this disclosure for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the disclosure illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate subcomponents, and anyone or more middle layers, such as a management layer, may be provided to communicatively couple to such sub-components in order to provide integrated functionality. Any components described in this disclosure may also interact with one or more other components not specifically described in this disclosure but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer readable storage medium; software transmitted on a computer readable transmission medium; or a combination thereof.

Moreover, the words “example” or “exemplary” are used in this disclosure to mean serving as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used in this description differently from one another as follows. Computer readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described in this disclosure. Furthermore, not all illustrated acts may be required to implement the methodologies in accordance with certain aspects of this disclosure. In addition, those skilled in the art will understand and appreciate that the methodologies could alternatively be represented as a series of interrelated states via a state diagram or events. Additionally, it should be appreciated that the methodologies disclosed in this disclosure are capable of being stored on an article of manufacture to facilitate transporting and transferring such methodologies to computing devices. The term article of manufacture, as used in this disclosure, is intended to encompass a computer program accessible from any computer-readable device or storage media.

What is claimed is:

1. A device comprising:
a memory having stored thereon computer executable components; and
a processor configured to execute the following computer executable components stored in the memory:
a sensing component that operatively couples to an interface component that is physically coupled to one or more sensors embedded in a container;
an analysis component that analyzes data gathered from the one or more sensors regarding attributes of a fluid in the container, and generates fluid status information; and
a communication component that wirelessly transmits the generated fluid status information to one or more remote devices.

2. The device of claim 1 is physically detachable from the interface component.

3. The device of claim 1, wherein the analysis component employs a Bayes network in connection with analyzing the fluid.

4. The device of claim 1, wherein the analysis component determines fluid level within the container, fluid pH, fluid contaminant level, age, and quality of the fluid.

5. The device of claim 1, being removeable from the interface and chargeable using at least one of solar, or inductive charging.

6. A system, comprising:
a container for storing fluid, the container comprising:
a plurality of fluid sensors vertically disposed along an interior of the container, and in physical contact with at least a portion of the fluid;
an interface that is physically coupled to the plurality of fluid sensors, and
a sensing device, configured to be physically attachable and detachable to the interface, the sensing device comprising:
a memory having stored thereon computer executable components; and
a processor configured to execute the following computer executable components stored in the memory:
a sensing component that operatively couples to the interface;
an analysis component that analyzes data gathered from the one or more sensors regarding attributes of the fluid in the container, and generates fluid status information; and
a communication component that wirelessly transmits the generated fluid status information to one or more remote devices.

7. The system of claim 6, wherein the device is physically detachable from the interface component.

8. The system of claim 6, wherein the analysis component employs a Bayes network in connection with analyzing the fluid.

9. The system of claim 6, wherein the analysis component determines fluid level within the container, fluid pH, fluid contaminant level, age, and quality of the fluid.

10. The system of claim 6, wherein the device is removeable from the interface and chargeable using at least one of solar, battery, or inductive charging.
